(19) United States
(12) Patent Application Publication
(10) Pub. No.: US 2026/0224457 A1
(43) Pub. Date: Aug. 6, 2026

(54) Pigmented Compositions Comprising Avobenzone

(71) Applicant: ELC Management LLC, Melville, NY (US)

(72) Inventor: Lisa Qu, Flushing, NY (US)

(21) Appl. No.: 19/046,224

(22) Filed: Feb. 5, 2025

(51) Int. Cl. A61K 8/29; A61K 8/35; A61Q 1/06; A61Q 1/08; A61Q 1/10; A61Q 1/12

(52) U.S. Cl. CPC A61K 8/29; A61K 8/35; A61Q 1/06; A61Q 1/08; A61Q 1/10; A61Q 1/12; A61K 2800/412; A61K 2800/43; A61K 2800/56

(57) ABSTRACT

Aqueous compositions comprising avobenzone and pigment grade titanium dioxide that has been surface-treated with calcium chloride. The surface treated titanium dioxide is able to stabilize avobenzone in aqueous compositions. The surface-treated titanium dioxide may be provided with an alginate coating to increase water solubility, while still protecting the avobenzone from the degrading effects of titanium dioxide.

### FIELD OF THE INVENTION

The present invention is in the field of topical compositions that comprise inorganic pigment and avobenzone. More specifically the compositions are aqueous makeup products, such as foundations and tinted moisturizers, that comprise avobenzone and titanium dioxide.

### BACKGROUND

Pigmented cosmetic products add color to the skin to conceal or highlight the skin's natural features. Such products include concealers, foundations, pressed powders, tinted moisturizers, blush, eyeshadow, lipstick and lip gloss. One of the more commonly used pigments in the cosmetics and personal care field is pigment grade titanium dioxide, which has a white, opaque appearance. For cosmetic purposes, pigment grade TiO₂ has a median particle size of about 200-300 nm, which is most effective for scattering visible light. Pigment grade TiO₂ for cosmetic use must be distinguished from “ultrafine” titanium dioxide, which has a median diameter of less than 100 nm, and which is commonly used in sunscreen products, in part, due to its transparency. The term “nano” is sometimes used to refer to grades of titanium dioxide that have a median diameter of 20-40 nm. Ultrafine and nano grades of TiO₂ should not be confused with pigment grades, which are well known to exhibit different properties. For example, pigment grade titanium dioxide is very effective at scattering visible light, but offers relatively little attenuation of UV radiation. In contrast, ultrafine TiO₂ is particularly good at attenuating UV radiation, especially UVB (290-320 nm) and UVA 2 (320-340 nm) through absorption (rather than scattering). Titanium dioxide occurs in various crystalline forms, rutile and anatase being the two most common. Rutile is the most thermodynamically stable form of the naturally occurring polymorphs of titanium dioxide. Throughout the remainder of this specification, unless otherwise stated, the phrase “titanium dioxide” will refer to pigment grade rutile.

The deleterious effects of ultraviolet radiation from the sun are well known. The term, “photoaging” refers to harmful effects induced in human skin by long term exposure to ultraviolet radiation. Various materials (collectively known as sunscreen filters) have been incorporated into topical compositions for the purpose of mitigating the effects of ultraviolet radiation on the skin. Consumer sunscreen products are intended to offer significant protection for skin that is continuously exposed to ultraviolet radiation for extended periods of time. However, in an effort to offer a measure of protection to consumers who experience short or intermittent periods of UV exposure, topical color cosmetics have also been formulated with UV filtering agents. One well known organic sunscreen filter is avobenzone. Avobenzone has been an important ingredient in sunscreen compositions since the 1970s. The preferred IUPAC name of avobenzone is 3-(4-tert-Butylphenyl)-1-(4-methoxyphenyl) propane-1,3-dione. Avobenzone is also known as butyl methoxydibenzoylmethane, while some common tradenames include: Parsol 1789, Eusolex 9020, Escalol 517, Neo Heliopan 357, and others. The peak absorbance wavelength of avobenzone is approximately 357 nm (nanometers), and the effectiveness of avobenzone at absorbing a broad range of UVA (320-400 nm) and UVB (290-320 nm) radiation is well attested. However, avobenzone can be thermodynamically unstable in aqueous formulations in the presence of certain other ingredients. This thermodynamic instability is separate from the well known tendency of avobenzone to be degraded by UV light (photo-instability).

Products that incorporate both avobenzone and titanium dioxide are known. In sunscreen products, both avobenzone and ultrafine TiO₂ are effective to filter UV radiation. In contrast, in color cosmetic products, avobenzone is used as a UV filter, while pigment grade TiO₂ is used for color, opacity and/or diffusion of visible light. Either type of product presents different challenges. For example, avobenzone is known to exhibit photo-instability, which is made worse by the photo-catalytic action of ultrafine titanium dioxide. On the other hand, in the presence of pigment grade TiO₂, avobenzone exhibits thermodynamic instability, so that breakdown of avobenzone occurs even in the dark. As a result of these issues, it is typical to treat the surface of TiO₂ particles with the aim of protecting avobenzone. A number of surface treatments have been tried, with differing levels of effectiveness. Silica and aluminum hydroxide are common treatments for titanium dioxide. Methicone, dimethicone, and polymethylmethacrylate are also used, but there remains a need for more fully effective methods of preserving the efficacy of avobenzone in aqueous compositions, in the presence of pigment grade titanium dioxide. To the best of our knowledge, calcium chloride has not been used to treat pigment grade titanium dioxide, to improve the thermodynamic stability of avobenzone in aqueous compositions.

U.S. Pat. No. 6,090,369 describes the combination of avobenzone, octyl-methoxycinnamate and titanium dioxide, wherein the presence of the titanium dioxide “serves to prevent the degradation of avobenzone when the composition is exposed to light.” The '369 disclosure is clear that the degradation of avobenzone that is being prevented is caused by photo-instability, not thermodynamic instability. Also, since titanium dioxide is a well-known catalyst for avobenzone photo-instability, a required element of the '369 disclosure is octyl-methoxycinnamate. Also, the titanium dioxide of '369 is no where said to be surface treated. This is unlike the present invention, which utilizes a surface-treated titanium dioxide to address the thermodynamic instability of avobenzone in aqueous systems, without the need for octyl-methoxycinnamate.

US2021-015726 discloses a cosmetic composition that includes a cosmetically acceptable carrier, 0.1% to 20% of avobenzone, and 0.1 wt % to 30 wt % of titanium dioxide particulate material, wherein, the titanium dioxide material is rutile, has a mean particle size of 350 nm to 500 nm, and the particles of the titanium dioxide have a silica coating. The compositions are said to address the problem of degradation of avobenzone caused by the presence of titanium dioxide, in storage (thermodynamic instability). In contrast, the present invention utilizes TiO₂ that has a median particle size of 200-300 nm, and a calcium chloride surface treatment.

### OBJECT OF THE INVENTION

A main object of the invention is to enhance the thermodynamic stability of avobenzone in the presence of pigment grade titanium dioxide, in aqueous environments.

### SUMMARY OF THE INVENTION

The foregoing objects of the invention are achieved in a topical pigmented composition that comprises avobenzone in combination with pigment grade titanium dioxide that has been surface-treated with calcium chloride. The water solubility of surface-treated TiO₂ may be enhanced by further providing the surface treated TiO₂ with a calcium alginate coating. The calcium alginate coating allows TiO₂ to be dispersed in water-based compositions, such as emulsions and gels.

### DETAILED DESCRIPTION

The dimensions and values disclosed herein are not to be understood as being strictly limited to the exact numerical values recited. Instead, unless otherwise specified, each such dimension is intended to mean both the recited value and a functionally equivalent range surrounding that value. Except where otherwise indicated, all numbers in this description indicating amounts or ratios of material or conditions of reaction, physical properties of materials and/or use are to be understood as modified by the word “about”. Unless otherwise specified, all amounts are by weight of the final composition.

As used herein, the term “comprise” means that a collection of elements is not necessarily limited to those explicitly recited.

### Avobenzone

A first main ingredient of the invention is avobenzone (C₂₀H₂₂O₃, CAS no. 70356-09-1). Avobenzone is readily oil soluble (for example, in isopropanol, decyl oleate, capric/caprylic triglyceride, dimethyl sulfoxide and castor oil), but not readily soluble in water (for example, only about 0.01 mg/L at 20° C.). Regulations specify the maximum concentration of avobenzone as 3% by weight of the composition. Compositions according to the present invention comprise a sunscreen-effective amount of avobenzone, such as 0.5% to 3% by weight of the composition. Preferably, compositions of the invention comprise 1% to 3%, by weight of the composition, more preferably, 2% to 3% by weight of the composition.

### Surface-Treated Titanium Dioxide

A second main ingredient of the invention is rutile pigment grade titanium dioxide (TiO₂), the surface of which has been treated with calcium chloride (CaCl₂)). A preferred median particle size of the TiO₂ is between 200 and 300 nm. Due the ability to scatter visible light, this size is optimal for the types of color cosmetic applications contemplated by the present invention. In aqueous solution, calcium chloride dissociates readily, and calcium ions adsorb onto the surface of titanium dioxide particles. In the present invention, the surface treated titanium dioxide comprises 0.1%-0.8% by weight of calcium ions, preferably 0.3% to 0.5%, such as 0.4% by weight of calcium ions.

Subsequent to surface treatment with calcium chloride, it is preferable if the surface-treated titanium dioxide is rendered hydrophilic. For example, the surface treated TiO₂ may be prepared with an alginate coating. Alginic acid, also known as algin, is an anionic polysaccharide ([C₆H₈O₆]ₙ) that occurs naturally in brown algae. Alginic acid is able to bind to the calcium ions of the surface-treated titanium dioxide, to form calcium alginate (CaCl₂H₁₄O₁₂). The alginate coating may be applied to the surface treated TiO₂ by treating the TiO₂ with sodium alginate (the sodium salt of alginic acid). The coated titanium dioxide is rendered hydrophilic, having excellent dispersibility in water. Preferably, the alginate coating comprises 1% to 3% by weight of the titanium dioxide particles.

Regulations specify the maximum concentration of titanium dioxide as 25% by weight of the composition. In compositions of the present invention, the concentration of surface treated, pigment grade TiO₂ may range from 1% to 25% by weight of the composition, and include all ranges encompassed within 1% to 25%, such as 1% to 20%, 2% to 10%, 3% to 5%, etc. Heavily pigmented products, such as concealer and foundation, may typically comprise 10% or more of pigment grade TiO₂. Lightly pigmented products, such as tinted moisturizers and lip gloss, may typically comprise 1% to 7% of pigment grade TiO₂, such as 1% to 6%, 1% to 5%, 1% to 4%, 2% to 5%, 2% to 6%, 2% to 7%, etc.

Multiple known methods of treating pigment grade titanium dioxide with calcium chloride and alginic acid may be applicable. One such method is the following.

### 1. Prepare a Suspension of TiO₂:

Disperse TiO₂ particles in deionized water or other appropriate solvent using mechanical stirring or ultrasonic homogenization to create a stable suspension.
A stabilizing agent may be added to prevent agglomeration of TiO₂ particles.
   2. Treat the Surface of TiO₂ with Calcium Chloride:
Slowly add a calculated amount of CaCl₂) solution to the TiO₂ suspension under constant stirring.
Adjust the pH of the suspension to be mildly alkaline (pH~8-9) using a base like sodium hydroxide. This facilitates the formation of calcium hydroxide on the TiO₂ surface.
Stir the suspension for at least one hour to allow uniform interaction and adsorption of calcium ions onto the TiO₂ surface.
Wash the surface-treated particles with deionized water to remove excess calcium chloride and by-products, then filter or centrifuge.
   3. Coat the Treated TiO₂ with Calcium Alginate:
Dissolve alginic acid in water to form a viscous solution.
Adjust pH to a slightly acidic range (pH~4-6) to ensure solubility.
Introduce the surface-treated TiO₂ into the alginic acid solution under continuous stirring.
Stir the mixture for a sufficient time (for example, 2-4 hours) to ensure complete reaction and uniform coating.

### 4. Post-Treatment:

Wash the alginate-coated TiO₂ particles with deionized water to remove unbound alginic acid and residual by-products.
Dry the coated particles in a vacuum oven or at a controlled low temperature (~40-60° C.) to preserve the coating integrity.
Optional Milling: If necessary, the dried particles are milled to achieve a uniform size distribution.

### Accelerated Aging Testing (8 Weeks @ RT, 40°, 50°)

Accelerated aging testing is intended to mimic long term storage conditions. A comparison was made between pigment grade titanium dioxide treated with calcium chloride and pigment grade titanium dioxide treated with aluminum hydroxide (control), to see if there is any difference in the effect of titanium dioxide on the thermodynamic stability of avobenzone. Test samples and control sample were prepared as shown in Table 1, in the form of oil-in-water emulsions. Among the calcium chloride samples, the level of treatment with alginic acid was also varied, as shown.

TABLE 1
Base Composition

| | aluminum | | | |
|---|---|---|---|---|
| | hydroxide/ | calcium | calcium | calcium |
| | 1% | chloride/ | chloride/ | chloride/ |
| | sodium | 1% | 2% | 3% |
| | alginate | sodium | sodium | sodium |
| Trade Name | (control) | alginate | alginate | alginate |
| water | q.s. | q.s. | q.s. | q.s. |
| octocrylene | 7.000 | 7.000 | 7.000 | 7.000 |
| ethylhexyl salicylate | 4.500 | 4.500 | 4.500 | 4.500 |
| homosalate | 2.000 | 2.000 | 2.000 | 2.000 |
| solvent(s) | 1.500 | 1.500 | 1.500 | 1.500 |
| emulsifier(s)/ | 4.000 | 4.000 | 4.000 | 4.000 |
| surfactants(s) | | | | |
| moisturizers/ | 11.990 | 11.990 | 11.990 | 11.990 |
| humectants/emollients | | | | |
| skin feel agent(s) | 4.000 | 4.000 | 4.000 | 4.000 |
| skin conditioning | 1.040 | 1.040 | 1.040 | 1.040 |
| agent(s) | | | | |
| skin active(s) | 4.600 | 4.600 | 4.600 | 4.600 |
| antimicrobial(s) | 0.660 | 0.660 | 0.660 | 0.660 |
| antioxidant(s) | 0.315 | 0.315 | 0.315 | 0.315 |
| preservative(s) | 0.148 | 0.148 | 0.148 | 0.148 |
| viscosity adjusters | 1.200 | 1.200 | 1.200 | 1.200 |
| rheology modifier(s) | 0.800 | 0.800 | 0.800 | 0.800 |
| iron oxides/algin | 0.634 | 0.634 | 0.634 | 0.634 |
| avobenzone | 3.000 | 3.000 | 3.000 | 3.000 |
| ¹MiyoAQUA White | 4.366 | | | |
| TSR | | | | |
| ²MiyoAQUA White | | 4.366 | | |
| C 1% | | | | |
| ³MiyoAQUA White | | | 4.366 | |
| C 2% | | | | |
| ⁴MiyoAQUA White | | | | 4.366 |
| C 3% | | | | |

¹MiyoAQUA White TSR (control)-pigment grade TiO₂ surface treated with 2.5% aluminum hydroxide and coated with 1% sodium alginate.
²MiyoAQUA White C 1%-pigment grade TiO₂ surface treated with 0.4% calcium chloride and coated with 1% sodium alginate.
³MiyoAQUA White C 2%-pigment grade TiO₂ surface treated with 0.4% calcium chloride and coated with 2% sodium alginate.
⁴MiyoAQUA White C 3%-pigment grade TiO₂ surface treated with 0.4% calcium chloride and coated with 3% sodium alginate.

MiyoAQUA White C has a median particle size of 204 nm. Samples were divided into groups for storage at 25° C., 40° C. or 50° C. (60%+5% relative humidity), for a total of 8 weeks. As is commonly done in the cometic arts, elevated temperature samples are used to simulate the effects of long term storage. All test samples and controls were stored in the dark, which simulates a typical situation where commercial products are filled in opaque packaging. The concentration of avobenzone in each sample was measured at time zero, and at 2 weeks, 4 weeks, 6 weeks, and 8 weeks.

### Results and Discussion

All three test samples (pigment grade TiO₂ with a calcium ions adsorbed onto the surface), at all three temperatures tested, showed no decrease in the concentration of avobenzone, within the sensitivity of the instrumentation. We conclude that avobenzone is thermodynamically stable in aqueous compositions, in the presence of pigment grade TiO₂ that has been surface treated with calcium chloride. Furthermore, as the results show, levels of avobenzone in the three test samples did not depend on the level of alginate coating (1%, 2% or 3%), so that algin may be used to improve water solubility while still ensuring that avobenzone will be protected from the degrading effects of titanium dioxide. In contrast, the control samples, which used TiO₂ with an aluminum hydroxide surface treatment and alginic acid, were not thermodynamically stable. The concentration of avobenzone decreased throughout the test period, so that by 8 weeks, avobenzone concentration decreased by 3.7%, 12.8% and 23.6% when stored in the dark, at 25° C., 40° C. and 50° C., respectively. Clearly, titanium dioxide with calcium chloride surface treatment is effective to stabilize avobenzone, and significantly more effective at stabilizing avobenzone than titanium dioxide with an aluminum hydroxide surface treatment.

What is claimed is:

1. A topical aqueous cosmetic composition comprising:
avobenzone; and
pigment grade titanium dioxide that has a median particle size that is between 200 and 300 nm, wherein the surface of the titanium dioxide has been treated with calcium chloride to adsorb calcium ions onto the surface of the titanium dioxide.

2. The composition of claim 1 wherein the concentration of avobenzone is between 0.5% and 3% by weight of the composition.

3. The composition of claim 1 wherein the concentration of surface treated titanium dioxide is between 1% and 25%.

4. The composition of claim 3 wherein the concentration of surface treated titanium dioxide is between 1% and 7%.

5. The composition of claim 1 wherein the surface-treated titanium dioxide is further treated with alginic acid to form a calcium alginate coating.

6. The composition of claim 5 in the form of a color cosmetic selected from the group comprising concealers, foundations, pressed powders, tinted moisturizers, blush, eyeshadow, lipstick and lip gloss.
